(19) United States
(12) Patent Application Publication
(10) Pub. No.: US 2008/0004610 A1
(43) Pub. Date: Jan. 3, 2008

(54) System for calculating IOL power

(76) Inventors: David Miller, Jamaica Plain, MA (US); Ramgopal Rao, Irvine, CA (US); Warren G. Young, San Diego, CA (US); Richard E.N. Lundberg, Newport Coast, CA (US); Edward Geraghty, Rancho Santa Margaita, CA (US)

Correspondence Address: DAVID R PRESTON & ASSOCIATES APC, 5850 OBERLIN DRIVE, SUITE 300, SAN DIEGO, CA, 92121, US

(21) Appl. No.: 11/821,952

(22) Filed: Jun. 26, 2007

Related U.S. Application Data
(60) Provisional application No. 60/817,351, filed on Jun. 30, 2006.

(51) Int. Cl. A61F 9/007

(52) U.S. Cl. 606/6

(57) ABSTRACT

The present invention relates to methods and apparatus to improve human ophthalmic surgery patient wellness and surgical procedural outcome of both indicated cataract surgery and elective surgeries with the implantation of permanent standard intraocular lens (IOL) and new technology intraocular lens (NTIOL). The present invention further relates to measurement of refraction intra-operatively to validate or obviate the pre-operative calculations and thus selection of the IOL or NTIOL and allow for a correction to be made intra-operatively before the permanent IOL or NTIOL is implanted. Specifically, several embodiments of the invention pertain to a disposable, temporary Proxy Lens apparatus that are used for in situ refractive measurements, an Insertion Tool apparatus to manipulate the Proxy Lens within the optical path for the refractive measurement and a Refractometer apparatus to perform the refractive measurements.

### RELATED DOCUMENTS

The present application claims benefit of priority to U.S. Provisional Application Ser. No. 60/817,351, filed Jun. 30, 2006, entitled “System for Calculating IOL Power” which is incorporate be reference herein in its entirety.

### FIELD OF THE INVENTION

The present invention relates generally to the field of opthalmology, and more particularly to methods and apparatus to improve human ophthalmic surgery patient wellness and surgical procedural outcome of both indicated cataract surgery and elective surgeries with the implantation of permanent standard intraocular lens (IOL) and new technology intraocular lens (NTIOL). The present invention further relates to measurement of refraction intra-operatively to validate or obviate the pre-operative calculations and thus selection of the IOL or NTIOL and allow for a correction to be made intra-operatively before the permanent IOL or NTIOL is implanted. Specifically, several embodiments of the invention pertain to a disposable, temporary Proxy Lens apparatus that are used for in situ refractive measurements, an Insertion Tool apparatus to manipulate the Proxy Lens within the optical path for the refractive measurement and a Refractometer apparatus to perform the refractive measurements.

### BACKGROUND

Cataract is a leading cause of blindness and is due to the opacification of the lens of the eye. The aging process is the leading cause of cataracts, though it may also occur with injuries, inflammation and other diseases. The World Health Organization estimates that more than 18 million people are affected worldwide and cataracts represent nearly 50% of world blindness (http://www.who.int/blindness/causes/priority). There are 28,000 new cases reported every day. Cataracts account for 25% of the vision loss of people over 65 years of age, and cataract surgery is the most common form of surgery in this age segment. The World Health Organization estimates that over the next 25 years, 20% of the population will be 65 years or older, leading to a significant increase in the incidence of cataracts. As one approaches 80 years of age, vision loss due to cataracts doubles to 50%. In the same time frame of 25 years from now, that segment of the population is expected to quadruple. The incidence of cataracts and cataract surgeries will grow at an extreme rate in the near future (www.worldhealth.net).

In a cataract surgery, the opaque lens is removed and a synthetic intraocular lens (IOL) is implanted in the eye. The refractive power of the IOL is chosen such a way that, ideally, the patient does not need any vision correction (from contact or spectacle lenses or Lasik, CK, etc.) after the surgery. Surgeons aim for emmetropia—i.e., no vision correction needed, but that this does not always occur. There are many IOL power calculation formulae in practice. These formulae use the parameters related to geometry (axial length and anterior chamber length etc.), lens properties and other “experience” factors to compute the desired power for the IOL. However, many factors such as inherent errors in the instrumentation that measure the geometry of the eye, measurement technique, individual differences in anatomy and uncertainty of final post-operative position of the IOL introduce errors in the calculated power of the IOL. In many cases patients experience “refractive surprise” rather than emmetropia. The current trend in cataract surgery is that a significant fraction of the surgeries involve implantation of an elective IOL. These New Technology IOLs (NTIOL) are accommodative and multi-focal. Another trend in ophthalmic surgery is that pre-cataract patients are increasingly electing NTIOLs for presbyopia correction prior to the appearance of cataracts. With increasing patient demand for the best possible post-operative vision after cataract extraction or the best possible correction of refractive error by clear lens extraction and refractive IOL implantation, the issue of predicting the ideal power of the IOL becomes central. Any elective surgery has its concomitant high patient expectations for surgical outcomes. Hereafter, the term IOL will refer to both IOL and NTIOL.

A summary of the key elements of possible errors in optimal IOL calculation has been reported (Hoffer, 2001; Kendall, 2001). These references indicate that the optimal IOL calculation revolves around three measurements:
1. The first is a measure of axial length, traditionally done by an ultrasound device. Over the years a number of corrections have been added to the ultrasound measurement, because the speed of the ultrasound beam varies with the density of the tissue traversed. Specifically, since the density of a cataract and the thickness of the lens will vary from patient to patient, the ultrasound speed will vary. Thus, some average correction must be used in the prediction formulae, which will lead to imprecise readings for cataracts at the ends of the bell shaped distribution of cataract densities. The axial length reading will also be imprecise because the ultra sound beam reflects off the sclera and thus, gives a reading from corneal apex to inner sclera, whereas the ideal axial length measurement should give the distance between corneal apex and the photo-receptor (retinal) plane. The distance from sclera to retinal surface, which constitutes a potential error in axial length measurement normally equals 0.20-0.30 mm. Thus, instead of using an ultrasound measurement, an optical measurement using light would be better. However, in cases of dense cataract, an optical technique cannot be used because the retina cannot be visualized (thus, the necessity of ultrasound).
2. The second is a measure of the power of the cornea. Since the corneal power is calculated before the surgery, the influence of the incision made to remove the cataract and implanting the IOL cannot be anticipated. Thus, the possible astigmatism created by the incision and it's healing may be a source of error in determining the ultimate precise power of the IOL. It should also be noted that the power of corneas that have undergone refractive surgery (i.e., laser alteration of the cornea or radial keratotomy) cannot be accurately measured and could lead to an error in the ultimate IOL power.
3. The third is a measure of the intended position of the IOL, i.e., its distance from the corneal apex. The values presently used vary for different IOL designs. These values are the result of a series of studies on post-operative eyes and thus represent an average value.

From the above discussion a number of conclusions become evident.
1. An optical measurement of axial length is more accurate than an ultrasound measurement. Such an optical measurement becomes possible in every case only after the cataract or clear lens has been removed. This suggests that the place to do the measurement is in the operating room after the cataract or clear lens is removed.
2. Pre-operative measurement of corneal power or total refractive power of the eye is subject to many errors and thus a method of determining the required power of the IOL, which is independent of such measurements, will be more desirable. This suggests that a method that measures the total refractive power of the eye during surgery would avoid the error inherent in corneal power measurement. This can be accomplished by inserting a lens of known power, called a Proxy Lens, at the same position where the IOL will be seated and then measuring the refractive power of the eye. From this measurement it would be possible to compute the power of the IOL that will have least post-operative refractive error.
3. The most accurate way to measure the distance between IOL and apex of the corneal surface is to make the measurement after an IOL is implanted and settled into position.
4. The best method for minimizing the post-operative refractive error should not involve a method that relies upon formulae and geometrical measurements made on the eye. It would be best to measure the refractive power of the eye with an IOL in place in the eye. This can only be done in surgery. Such a system and methodology will not depend on the accuracy of the geometrical measurements of the eye.

### SUMMARY OF THE INVENTION

The present invention provides methods and apparatus to improve human ophthalmic surgery patient wellness and surgical procedural outcome of both indicated cataract surgery and elective surgeries with the implantation of permanent standard intraocular lens (IOL) and new technology intraocular lens (NTIOL). The present invention further relates to measurement of refraction intra-operatively to validate or obviate the pre-operative calculations and thus selection of the IOL or NTIOL and allow for a correction to be made intra-operatively before the permanent IOL or NTIOL is implanted. Specifically, several embodiments of the invention pertain to a disposable, temporary Proxy Lens apparatus that may be used for in situ refractive measurements, an Insertion Tool apparatus that may be used to manipulate the Proxy Lens within the optical path for the refractive measurement and a Refractometer apparatus to perform the refractive measurements. Standardized methodology in intra-operative procedures with IOL or NTIOL implantation is described. The corrective procedures will reduce the probability that the patient will experience a “refractive surprise”, requiring post-operative corrective procedures to bring the patient back to emmetropia. This invention helps the surgeon achieve perfect vision in the patient by using previously non-existent intra-operative methods and apparatus in a manner described so that no additional corrective devices (from contact or spectacle lenses or Lasik, CK, etc.) need to be used by the patient during the post-operative phase.

These aspects of the invention, as well as others described herein, can be achieved by using the methods, articles of manufacture and compositions of matter herein. To gain a full appreciation of the scope of the present invention, it will be further recognized that various aspects of the present invention can be combined to make desirable embodiments of the invention.

### BRIEF DESCRIPTION OF THE DRAWINGS

FIG. 1 depicts an exploded view of the disposable apparatus indicating that the Insertion Tool has a different Proxy Lens that may be placed onto the Insertion Tool to accommodate a wide spectrum of refractive measurements.

FIG. 2 depicts the use of pairs of opposing visual marks on both the proximal and distal surfaces of the Proxy Lens to adjust the Proxy Lens plane perpendicular to the optical axis.

FIG. 3 depicts the use of a set of three points on either the proximal or distal surface of the Proxy Lens. The three points define an equilateral triangular (a=b=c) whose plane is planar with the Proxy Lens plane. The Proxy Lens is adjusted until the tetrahedron created with the apex of the point from the convergence of the two sensors has three isosceles triangles for its faces (a2=b2=c2).

FIG. 4 depicts a strain-gauge transducer attached to the distal end of the Insertion Tool handle. A readout of the transducer reflects the amount of pressure applied to the distal side of the Proxy Lens against the internal surface of the posterior face of the intraocular lens capsular bag.

FIG. 5 depicts flexion in the handle of the Insertion Tool apparatus by either using a soft material on the distal end of the handle, or by using channels or grooves cut into the convex (anterior) side of the distal end of the handle. Flexion is measured and reflects the amount of pressure applied to the distal side of the Proxy Lens against the internal surface of the posterior face of the intraocular lens capsular bag.

FIG. 6 depicts the premise of feature-based passive stereo photogrammetry used to determine depth information along the optical axis. Visual marks are used to determine the location. In this figure one mark (A) creates the conjoined pair for triangulation. Differences in the projected image location of A on the left image—A(xₗ,yₗ,zₗ)—and right image—A(xᵣ,yᵣ,zᵣ)—indicates the real world location A(x,y,z).

FIG. 7 depicts the apparatus used to measure total refraction in the eye (Refractometer), a disposable apparatus that is positioned within the path of the refractive measurement (Proxy Lens), and a carrier and handle on the disposable apparatus used to facilitate insertion and removal (Insertion Tool). A stereoscopic digital imaging system in the refractometer is used to locate the geometric center of the corneal dome, which is used as the reference for the optical axis.

### DETAILED DESCRIPTION OF THE INVENTION

### Definitions:

Throughout this application various publications are referenced. The disclosures of these publications are hereby incorporated by reference, in their entirety, in this application. Citations of these documents are not intended as an admission that any of them are pertinent prior art. All statements as to the date or representation as to the contents of these documents is based on the information available to the applicant and does not constitute any admission as to the correctness of the dates or contents of these documents.

Unless defined otherwise, all technical and scientific terms used herein have the same meaning as commonly understood by one of ordinary skill in the art to which this invention belongs. Generally, the nomenclature used herein and the surgical procedures in opthalmology, materials science, vision science, physics, electronics and computer software described below are well known and commonly employed in the art. Conventional methods are used for these procedures, such as those provided in the art and various general references. As employed throughout the disclosure, the following terms, unless otherwise indicated, shall be understood to have the following meanings. Where a term is provided in the singular, the inventors also contemplate the plural of that term. The nomenclature used herein and the surgical procedures described below are those well known and commonly employed in the art. Where there are discrepancies in terms and definitions used in references that are incorporated by reference, the terms used in this application shall have the definitions given herein.

Other technical terms used herein have their ordinary meaning in the art that they are used, as exemplified by a variety of technical dictionaries (for example, Chambers Dictionary of Science and Technology, Peter M. B. Walker (editor), Chambers Harrap Publishers, Ltd., Edinburgh, UK, 1999, 1325 pp). The inventors do not intend to be limited to a mechanism or mode of action. Reference thereto is provided for illustrative purposes only.

### INTRODUCTION

Throughout this application various publications are referenced. The disclosure of these publications are hereby incorporated by reference, in their entirety, in this application.

The present invention recognizes that it is desirable to have an in situ procedure that can be employed intra-operatively to validate the selection of the IOL that is to be implanted in the intraocular lens capsular bag as a corrective measure for cataracts or other forms of vision loss or impairments. A methodology to perform this validation is presented by several embodiments of the invention that define the apparatus required to measure total refraction in the eye. This methodology of measuring refraction of the entire eye is a more precise way of determining IOL power for the implantable IOL.

Total refraction of the eye is measured by use of a refractometer. Many refractometers, using conventional optics (as opposed to wave front aberrometers) have already been described in the patent literature and appear below:

| | | |
|---|---|---|
| 3036568 | (May 1962) | Stark |
| 3536383 | (October 1970) | Cornsweet |
| 3572909 | (March 1971) | Van Patten |
| 3762821 | (October 1973) | Bruning |
| 3930732 | (January 1976) | Holly |
| 4021102 | (May 1977) | Iizuka |
| 4353625 | (October 1982) | Nohda |
| 4367019 | (January 1983) | Kitao |
| 4372655 | (February 1983) | Matsumura |
| 4390255 | (June 1983) | Nohda |
| 4421391 | (December 1983) | Nohda |
| 4591247 | (May 1986) | Matsumura |
| 4620318 | (October 1986) | Kamiya |
| 4637700 | (January 1987) | Krueger |
| 4678297 | (July 1987) | Ishikawa |
| 4744648 | (May 1988) | Kato |
| 4755041 | (July 1988) | Ishikawa |
| 4761070 | (August 1988) | Fukuma |
| 4834528 | (May 1989) | Howland |
| 4859051 | (August 1989) | Fukuma |
| 5214456 | (May 1993) | Gersten |
| 5309186 | (May 1994) | Mizumo |
| 5500697 | (March 1996) | Fujieda |
| 5579063 | (November 1996) | Magnante |

### The Invention:

The present invention comprises the methods and apparatus for using a Proxy Lens apparatus (the Proxy Lens) of known refractive power, an Insertion Tool apparatus (the Insertion Tool) for holding and inserting the Proxy Lens in situ intra-operatively, and an intra-operative refractometer apparatus (the Refractometer) or any device that can measure refractive power—sphere, cylinder or other higher order refractive power—of the optical system of the eye. Specifically, it consists of:
1. An apparatus (Proxy Lens), which can be a disposable or a reusable item, is held within the optical path of the eye for the purpose of making refractive power measurements of the eye under surgery. The optical path may be external to the intraocular lens capsular bag (i.e., anterior chamber, iris surface, corneal apex) or within the intraocular lens capsular bag after the natural lens has been surgically removed.
2. An apparatus (Insertion Tool), which can be a disposable or a reusable item, to both hold and insert (or inject) the Proxy Lens into the optical path of the eye at the appropriate position during surgery for the purpose of making refractive power measurements of the eye.
3. An apparatus (Refractometer) to measure the refractive power of the eye during surgery. One described below uses stereoscopic digital imaging system or any other methodology to measure the refractive power of the eye. Other devices, such as optical surgical microscopes and wave front measurement systems, may also be used to measure the refractive power of the eye with the Proxy Lens held in the desired position.
4. A method for loading the Insertion Tool with the Proxy Lens.
5. A method of introducing the Insertion Tool with the attached Proxy Lens through an opening in the eye and holding the Proxy Lens at the desired position for the purpose of making refractive power measurements of the eye.
6. A method of calculating and selecting the refractive power of the IOL for the eye under surgery based on the refractive power measurements made with the Proxy Lens held at the desired position for the purpose of making refractive power measurements of the eye.
7. A method for verifying the refractive power of the eye with the Proxy Lens held at the desired position for the purpose of making refractive power measurements of the eye. This can be accomplished with the selection of a Proxy Lens of a calculated power. No refractive error should be present when the Proxy Lens power is same as the calculated power of the IOL. Such measurement will serve as a method of verification.
8. A method of correcting the calculated IOL power (as in item 6 above) based on the experience parameters such as healing factors, patient specific anatomy and surgeon related parameters. This will refine the prediction methodology so that the post-operative refractive error is minimized.

### The Proxy Lens:

The first unique element of the invention will be the use of an apparatus (Proxy Lens), which will simulate the IOL to be implanted. Measurements provided by the refractometer apparatus will precisely locate the position of the IOL in the post-operative eye. The Proxy Lens is small, disposable and hand held, and easily removable once inserted into the optical path of the eye. The physical characteristics of the Proxy Lens follow.
1. One embodiment of the Proxy Lens may be of any shape—plus, minus, cylindrical or multifocal.
2. Another embodiment of the Proxy Lens—of known power—is manufactured from a rigid or a soft (and thus foldable) transparent material.
3. Another embodiment of the Proxy Lens may also be inflatable with a gas or a liquid to make the refractive power adjustable. This also allows the Proxy Lens to be positioned against the internal surface of the posterior face of the intraocular lens capsular bag.
4. Another embodiment of the Proxy Lens has higher order aberration information manufactured within it to facilitate measurement and/or verification of higher orders of refraction.

Following the removal of the cataract or clear lens, the surgeon positions the Proxy Lens into the optical path for the refraction measurement. In one embodiment of the method, the Proxy Lens is positioned external to the intraocular lens capsular bag (i.e., anterior chamber, iris surface, corneal apex). In another embodiment of the method, the Proxy Lens in inserted through the initial incision used for the natural lens extraction and places it within the intraocular lens capsular bag. With the Proxy Lens held in position, a measurement of the refractive power of the intra-operative eye is made. The refractive measurement and the power of the Proxy Lens are then used to calculate the precise power of the intended IOL in order to give the patient maximal vision post-operatively.

The Proxy Lens inserted in the intraocular lens capsular bag has certain distinct advantages that are claimed.
1. The Proxy Lens approximates the in situ position of the natural lens and where the IOL will be implanted, reducing errors introduced during assumptions made with pre-operative predictive formulae. These errors include:
   a. anterior Chamber Depth and the final resting position of the IOL on the optical axis.
   b. decentration that may occurs as a result of implantation, and
   c. tilting of the IOL with respect to the lens plane perpendicularity relative to the optical axis.
2. If the position and orientation of the post-operative IOL can be more accurately assessed in longitudinal studies, these parameters can be used to position the Proxy Lens to approximate those parameters. This can then be used to more accurately adjust the selection of the correct power of the permanent IOL intra-operatively as addition measures of validation against the pre-operative predictive calculation of the refractive power.
3. The patient is conscious and is asked to fixate on a visual target in the optical field that represents the optical axis of the refractometer. An aphakic eye is unable to focus precisely on this visual target and lessens the accuracy of the visual field approximating the optical field. Inserting a Proxy Lens to increase visual acuity improves the patient's ability to assist with position the eye on the optical axis and making refractive measurements more accurate.

### The Insertion Tool:

The second unique element of the invention will be the use of an apparatus (Insertion Tool) to facilitate the insertion and removal of the Proxy Lens onto a position located on the optical axis so that a refractive measurement can be made. The physical characteristics of the Insertion Tool follow:
1. The Insertion Tool may have a permanent or a temporary Proxy Lens. If the Proxy Lens is permanent, the Proxy Lens and the Insertion Tool are manufactured as one unit and selection of the correct power Proxy Lens includes the attached Insertion Tool. If the Proxy Lens is temporary, the Insertion Tool will have on it a holding mechanism that positions the Proxy Lens in place for the insertion of the Proxy Lens onto the optical axis. The Insertion Tool in this case is a universal apparatus that may be used with Proxy Lens of different refractive powers.
2. In one embodiment of the Insertion Tool, a Light Emitting Diode (LED) or a fiber optic bundle is attached to the distal end of the handle on the distal side of the Proxy Lens landing to illuminate the posterior lens capsular bag, thus making it visible. This allows the position of internal surface of the position face of the intraocular capsular bag to be measured relative to the Proxy Lens.
3. In another embodiment of the Insertion Tool, an orifice manufactured in the distal end of the handle allows negative pressure to be introduced in the intraocular capsular bag. This negative pressure will force a constriction of the capsular bag and introduce surface based wrinkles that are visible on the internal surface of the position face of the intraocular capsular bag. These surface deformations will scatter and deflect light in patterns that yield more contrast and features. These features can then be detected in an area-based active stereo photogrammetry analysis to determine both shape and position of the posterior capsule.
4. In another embodiment of the Insertion Tool, the Proxy Lens is soft, thus allowing it to be folded. The Insertion Tool then is used as a holding device for the folded Proxy Lens and utilizes pneumatic pressure to inject the folded lens into the intraocular capsular bag where it is unfolded and placed into position.

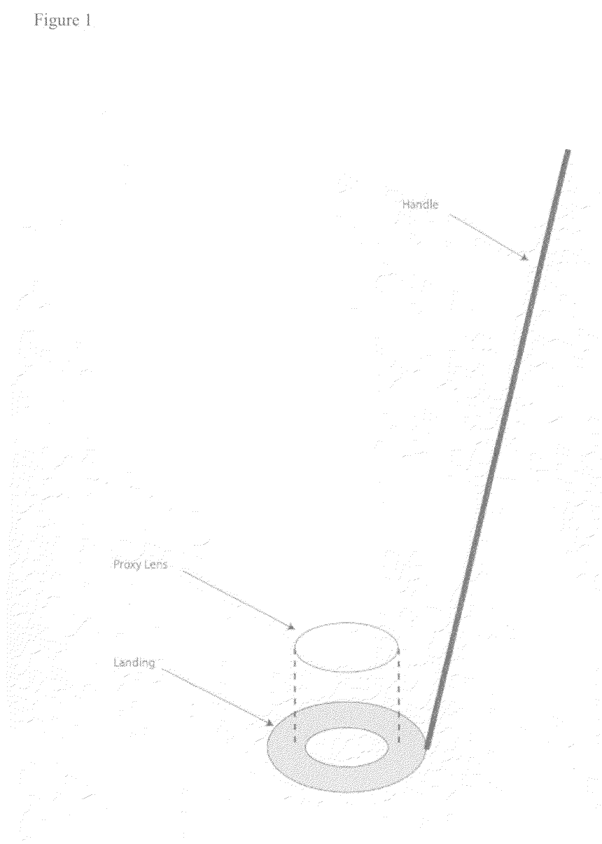

FIG. 1 shows the Insertion Tool and Proxy Lens. A handle is attached to the landing where the Proxy Lens is positioned. The surgeon uses the handle to manipulate Proxy Lens onto the optical axis.

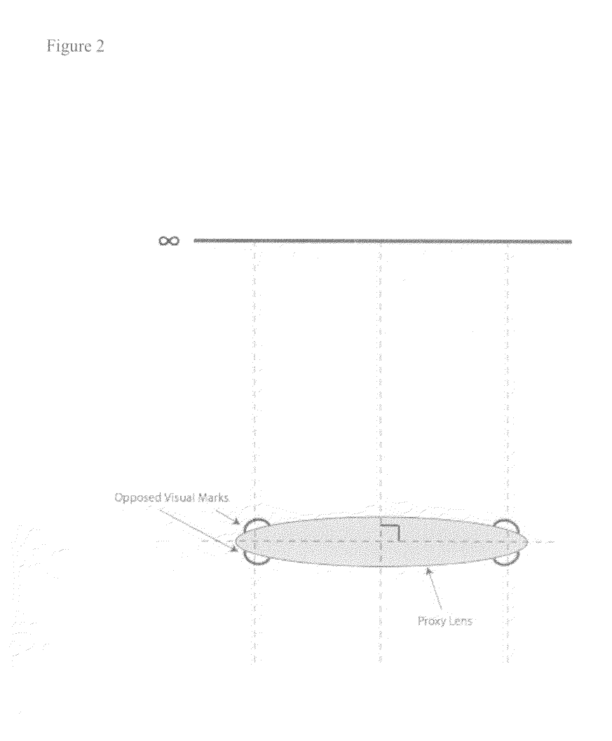

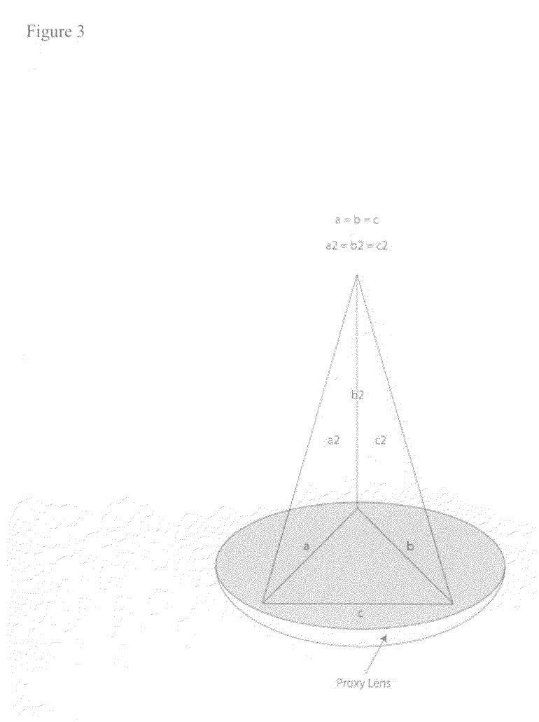

Several factors must be considered to mitigate factors that may lower the power and precision of the refractive measurement using the Proxy Lens and the Insertion Tool apparatus:
1. The plane of the Proxy Lens should be positioned perpendicular to the optical axis. If the Proxy Lens is not positioned in this manner, off-axis errors will be introduced during refractive measurements made with the refractometer. The orientation of the plane of the Proxy Lens can be adjusted with the implementation two methods that are claimed in this invention. Both methods assume that the fixation point is directly on the optical axis.
   A. Visual punctate marks are manufactured on the proximal and distal surface of the Proxy Lens as a set of pairs, and where the members of each pair of marks are directly in line with the optical axis of the Proxy Lens. The Proxy Lens is then held in place with the Insertion Tool apparatus and adjusted until each pair of opposing marks appear as one because they are directly on top of each other when viewed from a fixation point in the optical axis at infinity (FIG. 2). When this occurs visually, the Proxy Lens plane is considered to be perpendicular to the optical axis. Infinity is used for this theoretical argument because the refractometer apparatus of this invention uses non-orthogonal images for the stereo pair imaging. Because measurement at infinity is practically not possible, the alignment of the marks must be visualized from a fixation point that is off-axis by a distance from the center as determined by the location of the marks themselves.
   B. Visual punctate marks are manufactured on the proximal or distal surface of the Proxy Lens in a minimum set of three points near the peripheral edge of the surface. The location of the set of three points must define an equilateral triangle and the plane represented by these three points must be parallel to the plane of the Proxy Lens. The location of the set of three points will be measured by using the refractometer apparatus of this invention. One embodiment of this apparatus contains a stereoscopic digital imaging system. A distance measurement method, such as feature-based passive stereo photogrammetry, can then be used to calculate the location of each of the three points. This convergence of the two stereo cameras represents the apex of a tetrahedron. The planar equilateral triangle forms the base of the tetrahedron and the three triangular faces are formed by the two points of each segment of the planar equilateral triangle with the apex as the third point. The Proxy Lens is adjusted until each of the three faces of the tetrahedron is isosceles, indicating that the three points of the planar equilateral triangle are equidistance from the apex, and thus, the Proxy Lens plane is perpendicular to the optical axis (FIG. 3).
2. The position of the Proxy Lens relative to the posterior capsule should be controlled. Posterior capsule opacification (PCO) remains the most common long-term complication of cataract surgery (Apple et al, 1992; Kappelhof and Vrensen, 1992). In the most common forms of PCO, lens epithelial cells (LEC) migrate and proliferate between the posterior capsule and the IOL, forming monolayers and Elschnig pearls, leading to a decrease in visual acuity and loss of contrast sensitivity. The most common management option is capsulotomy using the neodymium-doped yttrium-aluminum-garnet (Nd:YAG) laser. The rate of incidence of PCO is declining due to new surgical tools and modern IOL design (Apple et al, 2001). Six factors were isolated leading to improved outcomes—three were surgery related and three were IOL related. Of the surgery related factors, depth fixation of the IOL intra-operatively is a critical factor where contact with the posterior side of the capsule decreases LEC migration and thus the incidence and severity of PCO. The contact of the two surfaces blocks migration of LEC from the equatorial side of the lens capsule (Peng et al, 2000). There are also reported adverse effects of YAG treatment for PCO (Billotte and Berdeaux, 2004). In this case, longitudinal studies of elderly patients with IOL implants and incidence of YAG capsulotomy at 5% rate vs. 20% rate decreases the incidence of intraocular pressure, glaucoma, macular edema and retinal detachment.

For these reasons, the depth fixation of the Proxy Lens relative to the internal surface of the posterior face of the intraocular lens capsular bag should be measured and controlled. The depth fixation of the Proxy Lens is critical to achieving the correct refraction measurement that reflects the optimal and desired position of the IOL.

Furthermore, the centration of the natural lens should also be considered. Studies have reported that the natural lens deviate 0.25 mm superiorly with a tilt of 6 degrees in the inferotemporal direction (Tscherning, 1898). Other studies report that IOLs are decentered about 0.64 mm superotemporally with a tilt of 6.75 degrees with the superonasal edges tipped forward after extracapsular cataract extraction (Auran et al, 1990). However, more recently, IOL decentration after phacoemulsification has improved to 0.14 to 0.34 mm with 2.06 to 4.88 degrees of tilt (Hayashi et al, 1997; Wang et al, 1998; Jung et al, 2000; Hayashi et al, 2001; Taketani et al, 2004).

For these reasons, the precise centration and tilt of the Proxy Lens relative to the optical axis should be measured and controlled. The centration of the Proxy Lens is critical to achieving the correct refraction measurement that reflects the optimal and desired position of the IOL. The tilt is any deviation from the planarity of the Proxy lens focal plane as measured perpendicular to the optical axis. This planarity is measured and enforced by the use of methods in this invention. Another embodiment of this invention introduces tilt as measured in post-operative conditions of implanted IOLs to simulate the final resting position and orientation of the IOL. This approximation of their final resting position and orientation will then give a more accurate intra-operative measure of refraction and allow for any changes in the selection of the permanent IOL intra-operatively.

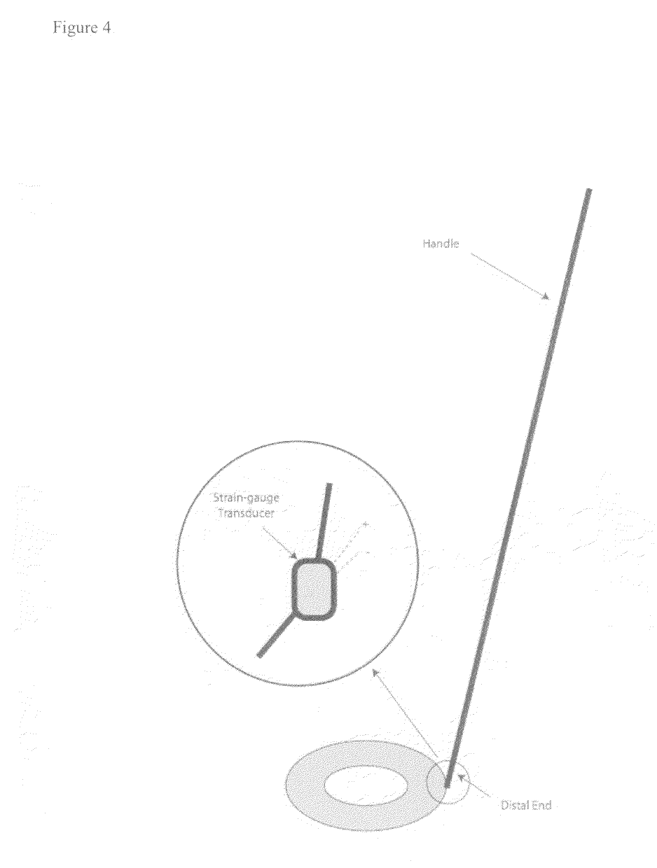

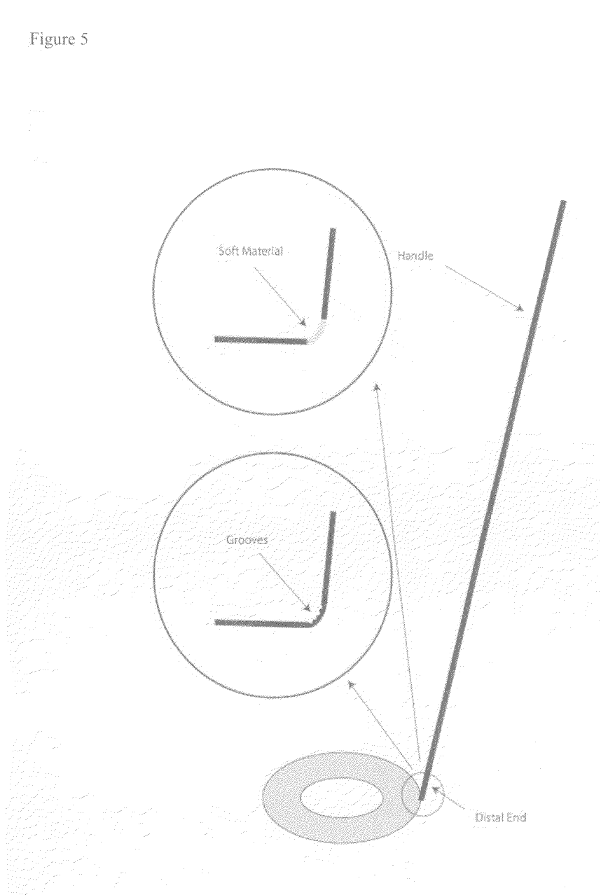

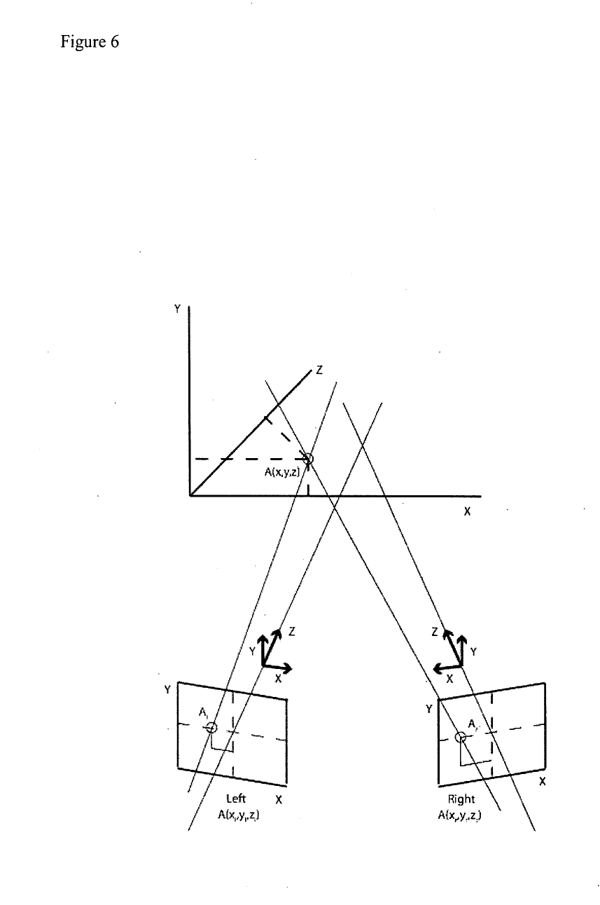

Current techniques and modern design of IOLs reduce errors with IOL fixation. However, depth fixation, centration and tilt of the Proxy Lens relative to the posterior surface of the intraocular lens capsular bag and the optical axis remain critical as a means to reduce post-operative complications and concomitant management. With the trend towards elective implantation of IOL, the use of the Proxy Lens and the intra-operative measurement of the refractive power will significant reduce post-operative complication. This is an important benefit of this invention. To facilitate the fixation of the Proxy Lens relative to the internal surface of the posterior face of the intraocular lens capsular bag, both A) physical and B) optical methods are claimed in this invention.
A. Physical methods encompass the measurement of proximity. Proximity measurements claimed in this invention utilize 1) strain-gauge transducers to measure pressure changes, and 2) flexion in the neck of the Insertion Tool apparatus handle.
   1. The Insertion Tool apparatus has a strain-gauge transducer at the distal end of the handle, just before the circular landing that holds the Proxy Lens (FIG. 4). As the landing that holds the Proxy Lens, or the Proxy Lens surface itself, contacts the internal surface of the posterior face of the intraocular lens capsular bag, the pressure increases as measured on an external readout. The pressure will reach an optimal zenith, which is determined empirically. The movement of the Insertion Tool apparatus is then halted for refractive measurement.
   2. The Insertion Tool apparatus has flexion engineered into the distal end of the handle, just before the circular landing that holds the Proxy Lens (FIG. 5). Flexion is introduced by two methods claimed in this for this apparatus:
      a. A different material at the distal end of the Insertion Tool handle that is softer and thus more pliable then the proximal end of the Insertion Tool handle,
      b. Channels or grooves cut onto the convex (anterior) side of the distal end of the Insertion Tool handle.
   As pressure increases in both cases, a distinct and measurable change in the angle is noted from the proximal end to the point of angle change, and from that point to the distal. The angle may be read by visualizing the components and geometry with the digital imaging system as one of the embodiments of the Refractometer apparatus. It may also be read by using a material that changes color when bent under pressure. The angle will reach an optimal zenith, which is determined empirically. The movement of the Insertion Tool apparatus is then halted for refractive measurement.
B. Optical methods encompass the measurement of 1) feature-based passive stereo photogrammetry and 2) area-based active stereo photogrammetry.
   1. In feature-based passive stereo photogrammetry, passive stereo vision (stereo photogrammetry) is used to calculate the 3-space position (depth) of the Proxy Lens by using corresponding 2-D points between the left and right images that are projections of the same physical point in the 3-D scene (stereo matching). By using the Refractometer apparatus in this invention that implements stereo vision, the geometric relationship between the two cameras and their intrinsic parameters is known from a calibration process. The 3-space coordinates of a point can be determined from the 2-D coordinates using epipolar geometry (Eric and Grimson, 1985; Chai, 1998). In these machine vision problems, the corresponding point in the first image of a conjoined pair for stereo matching is identified by searching along an epipolar line in the second image. These are computationally intense image processing tasks (Han et al, 2001; Deriche and Faugeras, 1990; Marapane and Trived, 1989; Weng et al, 1989; Eric and Grimson, 1985).
      With the Proxy Lens apparatus, the identification of the conjoined pair may be reduced to a much simpler computational task by manufacturing easily visualized discrete objects on the Proxy Lens itself and making highly confident assumptions of their locations on each of the stereo images (FIG. 6). The use of three locations will define the Proxy Lens focal plane, as is needed to determine the planarity of the lens in the optical path.
   2. When features are less distinct, such as on biological membranes, area-based active stereo photogrammetry can be used to determine position. The known position of the inner surface of the posterior side of the lens capsular bag will aid in approximating the position of the final resting position of the permanent IOL. This information would be used for the critical placement of the Proxy Lens for the measure of refraction. To enable visualization of the posterior surface for active stereo photogrammetry, several methods are claimed:
      a. Reflect a light from the posterior surface. As described earlier, this method is enabled with the use of the LED or the fiber optic bundle attached to the distal end of the handle on the Insertion Tool apparatus. The pattern of the reflected light can be a discrete point in which case conventional triangulation as in feature-based methods can be utilized. The pattern can also be a line or circle, so that additional information on the surface topography can be calculated by measuring deformations along the line scan axis.
      b. Use of a water soluble dye such as gentian violet or trypan blue ophthalmic solution to increase visibility of the posterior capsule.
      c. Improve visibility of the posterior surface by using negative pressure to introduce surface based wrinkles that are visible on the internal surface of the position face of the intraocular capsular bag. These surface deformations will scatter and deflect light in patterns that yield more contrast and features. These features can then be detected in an area-based active stereo photogrammetry analysis to determine both shape and position of the posterior capsule. As described earlier, this method is enabled with the use of an orifice manufactured in the distal end of the handle of the Insertion Tool apparatus.

A piano contact lens may also be used to neutralize any irregularity on the corneal surface (i.e., scratches or wrinkles). This will both help get an accurate refraction and allow better fixation on the part of the patient. The plano contact lens will also have marks on it in the same manner as the Proxy Lens to help visualize and determine its location on the optical axis with the same methodology mentioned above for the Proxy Lens.

### The Refractometer:

The third unique element of the invention will be use of an apparatus (Refractometer), which will perform the measure of refraction. The Refractometer will use a digital imaging system and an illuminated target. This is an architecture similar to many of the patents listed above in the Introduction. If the Proxy Lens is utilized to only get spherical and cylindrical first order numbers of aberration, the embodiment of the Refractometer need not be much different than what is in the prior art.

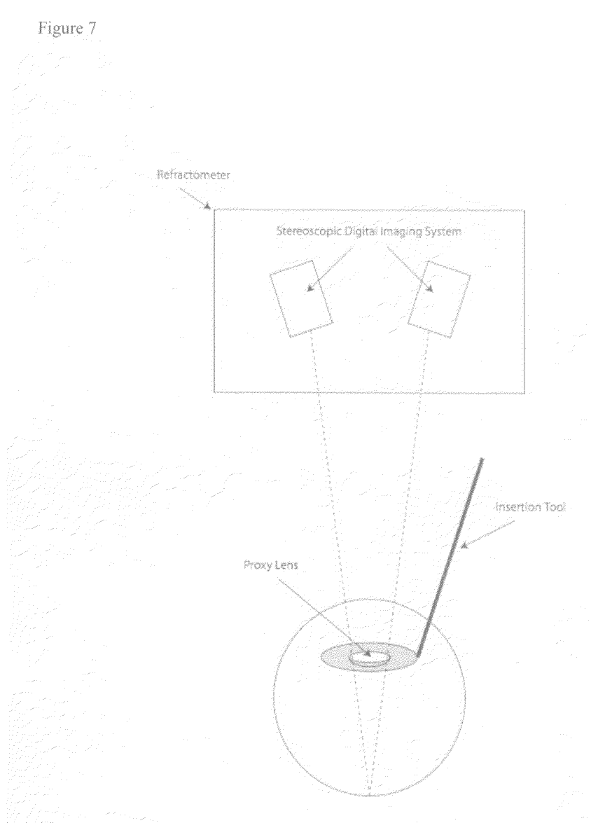

However, if accuracy of the placement of the Proxy Lens in 3-space is required, for example, to get higher order aberration parameters, or to more closely approximate the in situ position and orientation of the implanted IOL post-operatively, another embodiment of the Refractometer will require a stereoscopic digital imaging system to obtain three-dimensional information. This embodiment is unique compared to the above patents. The stereoscopic digital imaging system is used to locate the center (either the apex or the visual axis) of the “cornea dome” (i.e., geometric center of the cornea) and thus better relate the optical axis of the ocular optical system (the essential axis for a precise measurement) to a real anatomic reference point (FIG. 7). This is important in order to be able to reproduce and verify initial measurements. The stereoscopic digital imaging system will also be used for the various methods of this invention to locate position of objects, such as the Proxy Lens in 3-space.

The stereoscopic digital imaging system is referenced in another patent application (US 2005/0117118 A1, Jun. 2, 2005; PCT WO 03/030763 A1, Apr. 17, 2003).

### REFERENCES

1. Apple D J, Solomon K D and Tetz M R et al. Posterior capsule opacification. Surv Opthalmol. 1992; 37: pp. 73-116.
2. Apple D J, Peng Q and Visessook N et al. Eradication of posterior capsule opacification. Opthalmology. 2001; 108: pp. 505-518.
3. Auran J D, Koester C J, Donn A. In vivo measurement of posterior chamber intraocular lens decentration and tilt. Arch Opthalmol. 1990; 108: pp. 75-79.
4. Billotte C, Berdeaux G. Adverse clinical consequences of neodymium:YAG laser treatment of posterior capsule opacification. Journal of Cataract & Refractive Surgery. 2004; 30(10): pp. 2064-2071.
5. Chai J, De Ma S. Robust epipolar geometry estimation using genetic algorithm. Pattern Recognition Lett. 1998; 19: pp. 829-838.
6. Deriche R, Faugeras O. 2D-curves matching using high curvatures points: applications to stereovision. Proceedings of the 10th ICPR. Vol. 1, Atlantic City, 1990: pp. 240-242.
7. Eric W, Grimson L. Computational experiments with a feature based stereo algorithm. IEEE Trans. Pattern Anal. Mach. Intell. 1985; 7(1): pp. 17-34.
8. Han K P, Song K W, Chung E Y, Cho S J, Ha Y H. Stereo matching using genetic algorithm with adaptive chromosomes. Pattern Recognition. 2001; 34: pp. 1729-1740.
9. Hoffer K J. Important considerations for IOL calculations. (in) R B Wallace (ed) Refractive Cataract Surgery and Multifocal IOL's. Slack Publ. Thorofare, N J, 2001; pp. 37-53.
10. Kappelhof J P and Vrensen G F J M. The pathology of after-cataract; a minireview. Acta Opthalmol Suppl. 1992; 205: pp. 13-24.
11. Kendall C J. Ocular biometry for IOL calculations. (in) R B Wallace (ed). Refractive Surgery and Multifocal IOL's. Slack Publ. Thorofare, N J, 2001; pp. 9-21.
12. Jung C K, Chung S K, Baek N H. Decentration and tilt: silicone multifocal versus acrylic soft intraocular lenses. J Cataract Refract Surg. 2000; 26: pp. 582-585.
13. Hayashi K, Harada M, Hayashi H, et al. Decentration and tilt of poly-methyl methacrylate, silicone, and acrylic soft intraocular lenses. Opthalmology. 1997; 104: pp. 793-798.
14. Hayashi K, Hayashi H, Nakao F, Hayashi F. Anterior capsule contraction and intraocular lens decentration and tilt after hydrogel lens implantation. Br J. Opthalmol. 2001; 85: pp. 1294-1297.
15. Marapane S B, Trived M M. Region-based stereo analysis for robotic applications. IEEE Trans. System Man Cybernet. 1989; 19: pp. 1447-1464.
16. Peng Q, Visessook N and Apple D J et al. Surgical prevention of posterior capsule opacification. Part 3: intraocular lens optic barrier effect as a second line of defense. J Cataract Refract Surg. 2000; 26: pp. 198-213.
17. Taketani F, Matuura T, Yukawa E, Hara Y. Influence of intraocular lens tilt and decentration on wavefront aberrations. J Cataract Refract Surg. 2004; 30: pp. 2158-2162.
18. Tscherning M. Optique Physiologique; Dioptrique Oculaire—Fonctions de la Retined—Les Mouvements Oculaires et la Vision Binocular; Lecons professees a la Sorbonne. Paris, Carre & Naud. 1898; pp 62-63.
19. Wang M-C, Woung L-C, Hu C-Y, Kuo H-C. Position of poly(methyl methacrylate) and silicone intraocular lenses after phacoemulsification. J Cataract Refract Surg. 1998; 24: pp 1652-1657.
20. Weng J, Huang T, Ahuja N. Motion on structure from perspective views: algorithms, error analysis and error estimation. IEEE Trans. Pattern Anal. Mach. Intell. 1989; 11 (5): pp. 451-476.

We claim:

1. An apparatus to improve ophthalmic surgery patient wellness and surgical procedural outcome in both indicated cataract surgery and for elective surgeries using permanent, implantable intraocular lens devices, such as IOL (monofocal) and NTIOL (accommodative and multifocal), by facilitating the measurement of refraction proceeding removal of the natural lens and preceding the insertion of the IOL or NTIOL.

2. An apparatus of claim 1, wherein said apparatus comprises a Proxy Lens, which can be a disposable or a reusable item and is held within the optical path of the eye for the purpose of making refractive power measurements of the eye under surgery.

3. An apparatus of claim 1, wherein said apparatus comprises an Insertion Tool which can be a disposable or a reusable item, to both hold and insert (or inject) the Proxy Lens into the optical path of the eye at the appropriate position during surgery for the purpose of making refractive power measurements of the eye.

4. An apparatus of claim 1, wherein said apparatus comprises a Refractometer to measure the refractive power of the eye during surgery using stereoscopic digital imaging system or any other methodology to measure the refractive power of the eye.

5. The apparatus of claim 2, wherein said Proxy Lens may be one of any shape (plus, minus, cylindrical or multifocal), any transparent material (rigid or soft), inflatable with an injectable medium, whereby the lens power is adjustable with different volumes of medium (gas or liquid), can contain higher order aberration information to correct for other abnormal vision conditions (astigmatism), and be placed at various locations in the optical path for measurement and comparison to the permanent IOL or NTIOL.

6. The apparatus of claim 2, wherein said Proxy Lens position in the intraocular lens capsular bag can approximate the in situ position of the natural lens for reduced errors during measurement of refraction.

7. The apparatus of claim 2, wherein said Proxy Lens optimal diameter when inserted into the intraocular lens capsular bag is equal to or less then the size of a standard surgical incision (i.e., 3 mm) or larger than the standard surgical incision (i.e., 3 mm) and is then folded in this instance prior to insertion into the intraocular lens capsular bag.

8. The apparatus of claim 2, wherein said Proxy Lens contains visual markings as a pair of punctate marks on opposing surfaces (proximal and distal) on the surface that are used to orient the Proxy Lens by adjusting the plane of the Proxy Lens until each pair of opposing marks appear as one because they are directly on top of each other in the optical axis when viewed from a fixation point on the optical axis at infinity.

9. The apparatus of claim 2, wherein said Proxy Lens contains visual markings as three punctate marks on the peripheral edges of the surface (proximal or distal) and form an equilateral triangle on the surface of the Proxy Lens, and whose plane represented by these three marks must be parallel to the plane of the Proxy Lens.

10. The apparatus of claim 3, wherein said Insertion Tool has a landing on which a rigid Proxy Lens is attached (permanently or temporarily with the use of a holding mechanism) and a handle that the surgeon uses to hold and manipulate the Insertion Tool and thus the position of the Proxy Lens in the optical path for the measurement of refraction.

11. The apparatus of claim 3, wherein said Insertion Tool has physical devices on the distal end, such as a Light Emitting Diode (LED), fiber optic bundle, or an orifice that introduces a negative pressure in the intraocular capsular bag to make the internal surface of the position face of the intraocular capsular bag more visible, thereby aiding in the measurement of refraction by increasing the illumination of the internal surface of the position face of the intraocular capsular bag.

12. The apparatus of claim 3, wherein said Insertion Tool facilitates insertion of the Proxy Lens by injection with a pneumatic piston to apply external pressure thus moving the folded Proxy Lens into the intraocular lens capsular bag.

13. The apparatus of claim 3, wherein said Insertion Tool maintains the position of the plane of the Proxy Lens as perpendicular to the optical axis to increase the accuracy of the measurement of refraction.

14. The apparatus of claim 3, wherein said Insertion Tool maintains the 3-space position of the Proxy Lens for refractive measurement and can be located adjacent to the internal surface of the posterior face of the intraocular lens capsular bag to reflect the natural location of the natural lens and increases the accuracy of the measurement of refraction.

15. The apparatus of claim 4, wherein said Refractometer uses stereoscopic digital imaging to determine the triangulation of the Proxy Lens for planarity.

16. The apparatus of claim 4, wherein said Refractometer uses a plano contact lens to neutralize any irregularity on the corneal surface and improve the measurement of refraction.

17. The apparatus of claim 4, wherein said Refractometer determines the 3-space position of the Proxy Lens and renders information on the Anterior Chamber Depth that indicates the fixation along the optical axis, centration relative to the optical axis, and the tilting of the plane of the Proxy Lens relative to the optical axis.

18. The apparatus of claim 4, wherein said Refractometer determines the 3-space position of the Proxy Lens with a physical or optical apparatus.

19. The apparatus of claim 17, wherein said physical apparatus employs the use of strain gauge transducers on the distal end of the Insertion Tool handle that measure the pressure exerted by the posterior capsule on the Proxy Lens.

20. The apparatus of claim 17, wherein said optical apparatus employs the use of softer material on the distal end of the Insertion Tool handle that has channels or grooves introduced on the convex (anterior) side of the Insertion Tool handle where flexion is measured by visualizing the components and geometry with the Refractometer apparatus.

21. The apparatus of claim 17, wherein said optical apparatus employs the use of material that changes color when bent under pressure.

22. The apparatus of claim 17, wherein said optical apparatus employs the use of feature-based passive or active stereo photogrammetry to measure distance in the optical axis.

23. The apparatus of claim 21, wherein said passive stereo photogrammetry measurement of distance with a stereoscopic microscope is enhanced with visually detectable markings on the Proxy Lens distal surface that is adjacent to the internal surface of the posterior face of the intraocular lens capsular bag.

24. The apparatus of claim 21, wherein said active stereo photogrammetry measurement of distance is facilitated with light emitters from the distal end of the handle of the Insertion Tool.

25. The apparatus of claim 21, wherein said active stereo photogrammetry measurement of distance is facilitated by introducing negative pressure in the intraocular capsular bag.

26. The apparatus of claim 21, wherein said passive or active stereo photogrammetry measurement of distance with a stereoscopic microscope is enhanced with the use of water-soluble dyes to visualize the internal surface of the posterior face of the intraocular lens capsular bag.

27. The apparatus of claim 26, wherein said water-soluble dye is gentian violet.

28. The apparatus of claim 26, wherein said water-soluble dye is trypan blue ophthalmic solution.
